(19) United States
(12) Patent Application Publication
(10) Pub. No.: US 2024/0307069 A1
(43) Pub. Date: Sep. 19, 2024

(54) INTERACTIVE INDICATOR SENSING TOURNIQUET

(71) Applicant: University of Kentucky Research Foundation, Lexington, KY (US)

(72) Inventors: Brittany E. Levy, Lexington, KY (US); Jennifer T. Castle, Lexington, KY (US); Grant M Levy, Lexington, KY (US)

(21) Appl. No.: 18/441,876

(22) Filed: Feb. 14, 2024

Related U.S. Application Data
(60) Provisional application No. 63/484,894, filed on Feb. 14, 2023.

(51) Int. Cl. A61B 17/132; A61B 17/00; A61B 90/00

(52) U.S. Cl. CPC A61B 17/1327; A61B 90/08; A61B 2017/00022; A61B 2017/00115; A61B 2090/0807

(57) ABSTRACT

A “smart” tourniquet device includes a fastening strap, a tourniquet tightener and a control module carried on the fastening strap. The control module includes a controller, an information display, an indicator and a power source. The “smart” tourniquet device may be used in a method of occluding blood flow through a body part, to reduce trauma related morbidity and mortality.

### RELATED APPLICATIONS

This document claims the benefit of U.S. Provisional Patent Application Ser. No. 63/484,894, filed on Feb. 14, 2023, the full disclosure of which is incorporated herein by reference.

### TECHNICAL FIELD

This document relates generally to a new and improved “smart” tourniquet as well as to a method of use of that tourniquet.

### BACKGROUND

Tourniquets are used in emergency settings for life threatening hemorrhage in extremities. Application of a tourniquet requires a specific order of steps to achieve appropriate and effective application, whereby arterial blood flow is occluded to stop hemorrhage. With over 30%-40% of trauma related deaths attributable to hemorrhage, appropriate tourniquet application and early hemostasis has a significant role in reducing trauma related morbidity and mortality. In order to train lay people and prehospital emergency responders on tourniquet use, multiple public health education initiatives have been launched including “Stop the Bleed”. This is a nationwide effort to increase awareness and familiarity with efficacious tourniquet application. However, even in populations where this training is provided, still up to 27% of tourniquets are mal placed, with a meaningful proportion of those patients experiencing harm due to delay in hemorrhage control.

This document relates to a new and improved tourniquet device that provides real time audio and visual feedback to the user adapted to ensure proper use and application of the tourniquet device. This is accomplished in two feedback cycles. The first feedback cycle monitors and guides the user in the proper application of the tourniquet device to a patient while the second feedback cycle detects arterial occlusion in the patient, alerts the user when arterial occlusion has been achieved and initiates a timer so that the duration of the arterial occlusion is known to the subsequent care-giver.

### SUMMARY

In accordance with the purposes and benefits set forth herein, a new and improved tourniquet device is provided. That tourniquet device comprises: (a) a fastening strap, (b) a tourniquet tightener spanning at least a portion of the fastening strap, and (c) a control module carried on the fastening strap, the control module including a controller, an information display, and a power source.

In one or more of the many possible embodiments, the tourniquet device may include an indicator. The indicator may comprise a speaker for providing an audio indication, an indicator light for providing a visual indication or both a speaker and an indicator light for providing both an audio indication and a visual indication of a predetermined condition. In some embodiments, the indicator may be integrated into or be a part of the information display.

In one or more of the many possible embodiments of the tourniquet device, the tourniquet tightener includes a tightening strap, connected across at least a portion of the fastening strap and a windlass adapted for winding the tightening strap to tighten the fastening strap around a body part and occlude blood flow through the body part of an individual.

In one or more of the many possible embodiments of the tourniquet device, a fastener is carried on the fastening strap. The fastener may include a hook and loop fastener carried on the first end of the fastening strap and a fastening ring carried on the second end of the fastening strap. The hook and loop fastener may be conductive and the controller may be adapted to (a) monitor the conductive hook and loop fastener and (b) provide a signal when the conductive hook and loop fastener has been properly fastened and the circuit between the hooks and the loops has been completed or closed.

In at least some embodiments of the tourniquet device, the tourniquet device further includes at least one sensor adapted to detect occlusion of blood flow through the body part about which the tourniquet device has been applied. That sensor may comprise a pulse sensor, an oxygen sensor or a pulse and oxygen sensor.

In at least some embodiments of the tourniquet device, the controller is adapted to monitor a duration of time since blood flow occlusion and indicate the time duration on the information display.

In some embodiments, indicia is carried on the fastening strap. That indicia is adapted to indicate which side of the fastening strap is to be oriented toward the heart of the individual to which the tourniquet device is applied. In some embodiments, the tourniquet device further includes a windlass retainer carried on the fastening strap that is adapted to hold the windlass in a wound condition so as to maintain tightening pressure and blood occlusion once blood occlusion has been achieved.

In accordance with an additional aspect, a tourniquet device, comprises: (a) a fastening strap adapted for positioning around a body part, (b) a tourniquet tightener carried on the fastening strap, (c) a conductive fastener carried on the fastening strap, and (d) a control module carried on the fastening strap, the control module including a controller adapted to monitor the conductive fastener and provide a signal when the conductive fastener has been properly fastened.

In accordance with yet another aspect, a method of occluding blood flow through a body part using a tourniquet device, comprises steps of: (a) positioning a fastening strap of the tourniquet device around the body part and fastening the fastening strap, (b) tightening the fastening strap around the body part to occlude blood flow past the fastening strap, (c) detecting, by at least one sensor, when blood flow is occluded, (d) alerting, by controller, a user to quit tightening the fastening strap once blood flow is occluded and (e) initiating, by the controller, a timer to monitor a duration of time since blood flow occlusion was achieved.

In one or more possible embodiments of the method, the method further includes one or more of the following steps: (a) detecting, by the controller, when the fastening strap is properly fastened, and (b) signaling, by the controller, when the fastening strap is properly fastened. Signaling when the fastening strap is properly fastened may include (a) providing an audio indication signal, (b) providing a visual indication signal or (c) providing both an audio indication signal and a visual indication signal.

In one or more of the many possible embodiments of the method, the tightening of the fastening strap includes winding, by windlass, a tightening strap, carried on the fastening strap, to tighten the fastening strap around the body part. Still further, the winding, by the windlass, may include turning a rod to twist and shorten the tightening strap.

In one or more embodiments of the method, the method includes the step of providing a windlass retainer on the tourniquet device and engaging the rod with the windlass retainer to hold the rod in a wound condition once the blood flow has been occluded. In one or more embodiments, the method includes the step of displaying the duration of time since blood flow occlusion on a display carried on the tourniquet device.

In another possible aspect, the method includes the steps of: (a) monitoring and guiding, by a controller, a user in proper application of the tourniquet device to the body part, (b) detecting, by the controller, arterial occlusion in the body part, and (c) alerting, by the controller, when the arterial occlusion has been achieved. Still further, the method may include (d) initiating, by the controller, a timer to time a duration of the arterial occlusion.

In the following description, there are shown and described several embodiments of the tourniquet device and related method of occluding blood flow through a body part using the tourniquet device. As it should be realized, the device and method are capable of other, different embodiments and their several details are capable of modification in various, obvious aspects all without departing from the device and method as set forth and described in the following claims. Accordingly, the drawing and descriptions should be regarded as illustrative in nature and not as restrictive.

### BRIEF DESCRIPTION OF THE DRAWING FIGURE

The accompanying drawing figures incorporated herein by reference and forming a part of the specification, illustrate several aspects of the new and improved tourniquet device and method adapted for providing real time audio and visual feedback to the user to ensure proper use and application of the tourniquet device.

FIG. 1 is a schematic perspective view of one possible embodiment of the tourniquet device.

FIG. 2A is a perspective view illustrating how the tourniquet device is initially placed around the body part, in this instance a thigh, through which blood flow is to be occluded.

FIG. 2B is a similar view illustrating how the fastener is fastened to secure the tourniquet device around the thigh.

FIG. 2C is a similar view illustrating how the windlass is wound (note action arrows) to twist the tightening strap and thereby tighten the fastening strap until blood flow though the thigh is occluded.

FIG. 2D is a similar view illustrating how the windlass is secured to maintain occlusion of blood flow.

Reference will now be made in detail to the present preferred embodiments of the device.

### DETAILED DESCRIPTION

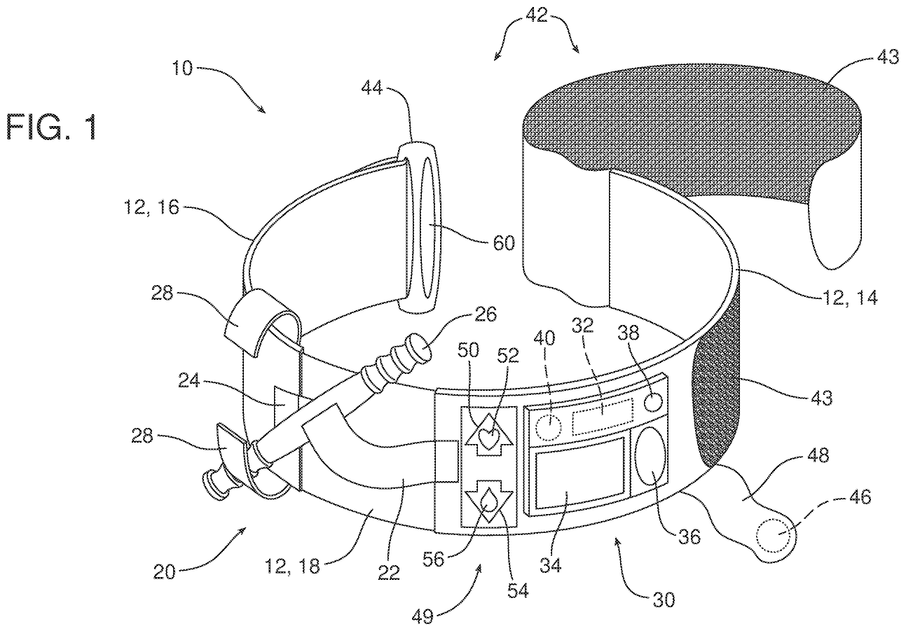

Reference is now made to FIG. 1 illustrating one possible embodiment of the new and improved “smart” tourniquet device 10. The tourniquet device 10 includes a fastening strap 12 having a first end section 14, a second end section 16, and an intermediate section 18 connecting the first end section and the second end section. The intermediate section 18 may be made from a collapsible or elastic material while the first and second end sections 14, 16 may be made from a non-elastic material.

A tourniquet tightener, generally designated by reference numeral 20, spans at least a portion of the intermediate section 18. In the illustrated embodiment, the tourniquet tightener 20 includes (a) a tightening strap, having a first section 22 connected to the first end section 14, a second section 24 connected to the second end section 16, and (b) a windlass 26, in the form of a short rod, adapted for winding or twisting the two sections 22, 24 of the tightening strap together to tighten the fastening strap around a body part and occlude blood flow through the body part of an individual to stop hemorrhaging. A windlass retainer 28 may be carried on the fastening strap 12. Such a windlass retainer 28 may comprise a C-shaped clip adapted, once blood occlusion has been achieved, to hold the windlass/rod 26 in a wound condition to maintain the necessary pressure to ensure that blood flow occlusion is maintained over time.

The tourniquet device 10 further includes a control module, generally designated by reference numeral 30, carried on the fastening strap 12. The control module 30 includes a controller 32, an information display 34, an optional indicator 36, an actuator switch 38, which may take any form known in the art, and a power source 40. The controller 32 may comprise, for example, a computing device such as a dedicated microprocessor operated by appropriate hardware instructions or an electronic control unit (ECU) operated in accordance with appropriate software instructions.

The indicator 36 may be an audio indicator, such as a speaker, a visual indicator such as an indicator light or lamp or audio and visual indicators of any type known in the art to be useful for such a purpose. In some embodiments, the information display/viewing screen 34 may serve as or incorporate the indicator. Such an indicator is adapted to provide audio and/or visual feedback to a user of the device 10 upon detection of a predetermined condition. Such a condition may relate to the proper application and use of the tourniquet device 10, such as the proper fastening of the fastening strap 12 around the body part or the complete occlusion of the blood flow past the fastening strap and through the body part or extremity in which the hemorrhage is found. This audio and/or visual feedback allows the user to confidently use the tourniquet device 10 in the proper and most effective manner to control hemorrhage conditions and save lives.

A fastener 42 is carried on the fastening strap 12. In the illustrated embodiment, the fastener 42 comprises a cooperating conductive hook and loop fastener 43 carried on the first end section 14 and a fastening ring 44 connected to the second end section 16. The controller 32 is adapted to monitor the conductive fastener 42 and provide a signal when the conductive fastener has been properly fastened.

The tourniquet device 10 also includes at least one sensor 46 adapted to detect occlusion of blood flow past or distal to the fastening strap 12 and through the body part. That sensor 46 may be carried on a flap of material 48 depending from the first end section 14 as shown in the drawing figure. The sensor 46 may comprise a pulse sensor, an oxygen sensor or both a pulse sensor and an oxygen sensor. The sensor 46 may be a photoplethysmogram sensor. For example, blood occlusion has occurred when diastolic pressure is no longer readable by the pulse sensor 46. Other sensors appropriate for detecting blood flow occlusion may also be utilize instead of or in addition to a pulse sensor and/or an oxygen sensor.

The controller 32 is also adapted to monitor a duration of time since blood flow occlusion and indicate that duration of time on the information display/viewing screen 34. This information is vital to the medical staff that will subsequently treat the patient at the hospital or clinic.

The tourniquet device 10, illustrated in the drawing figures, also includes indicia 49 carried on the fastening strap 12. In the illustrated embodiment, the indicia 49 comprises a first arrow 50, including a heart outline 52, pointing in a first direction and a second arrow 54, pointing in a second opposite direction. The second arrow 54 may include an outline of a drop of blood 56. The indicia 49 indicates which side of the fastening strap 12 should be oriented toward the heart and which side of the fastening strap should be oriented away from the heart when the tourniquet device is being applied on the body part of an individual. Note the second arrow 54 points toward the second side of the fastening strap 12 from which the flap 48 and sensor 46 depend.

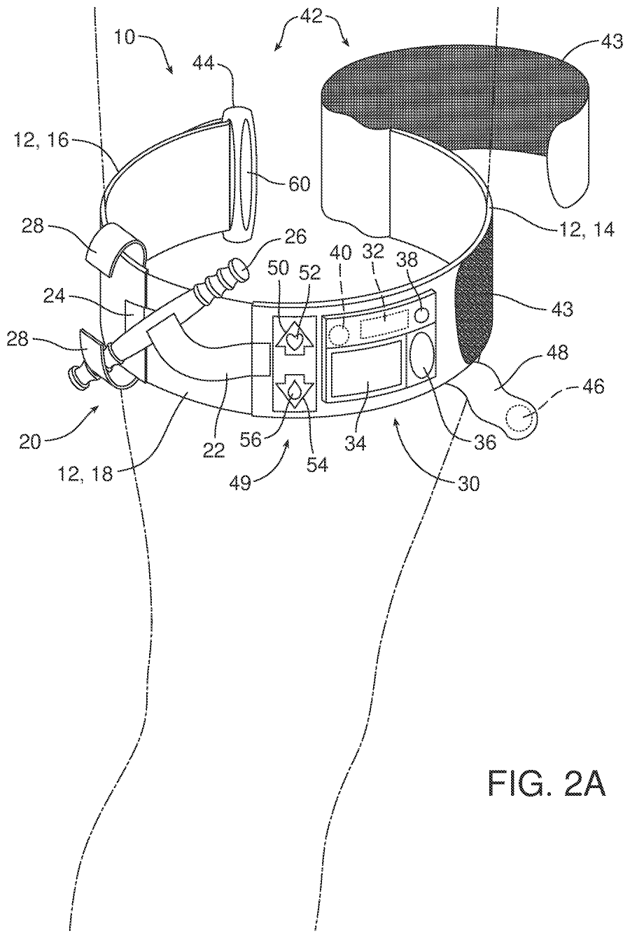

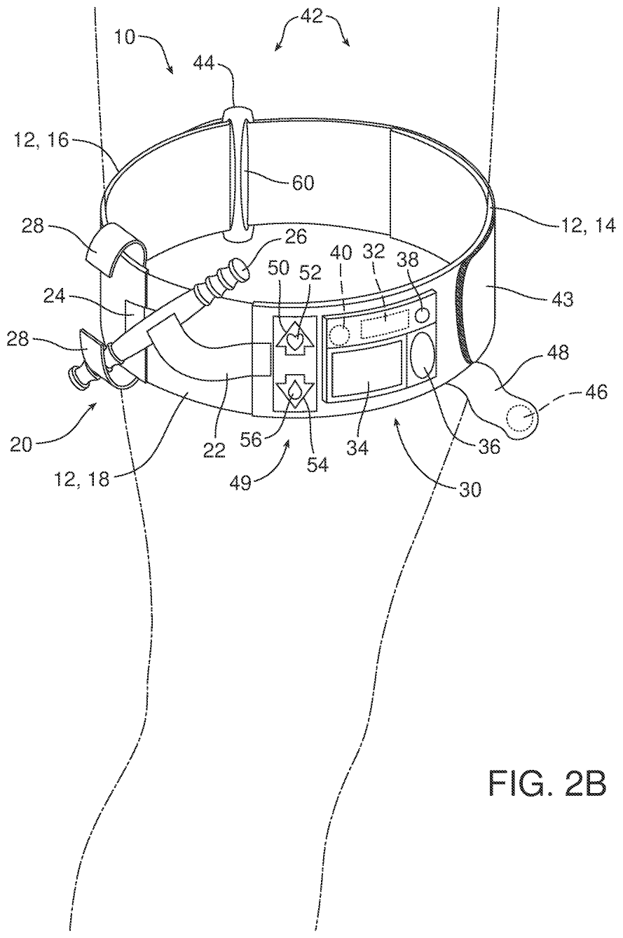

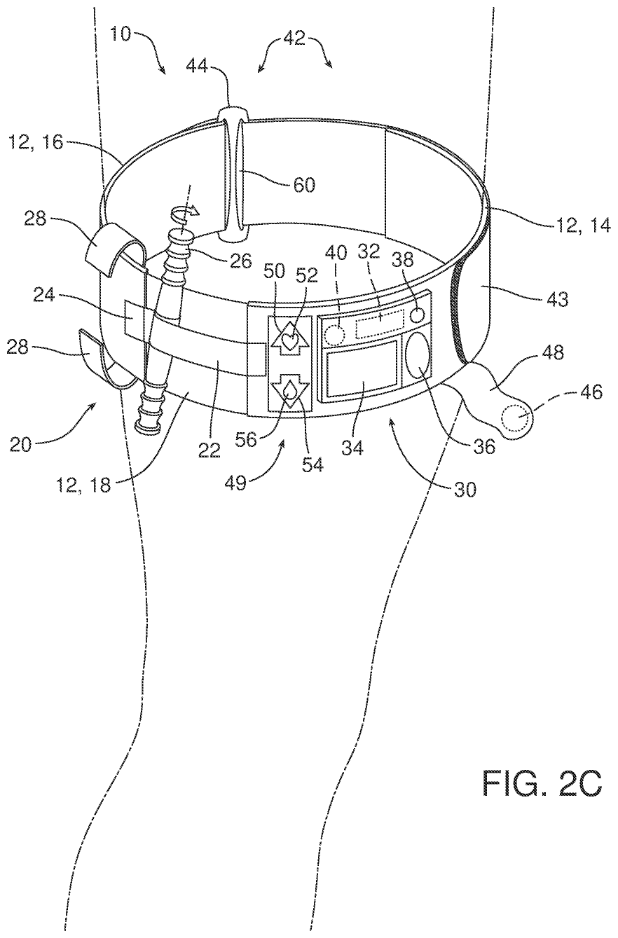

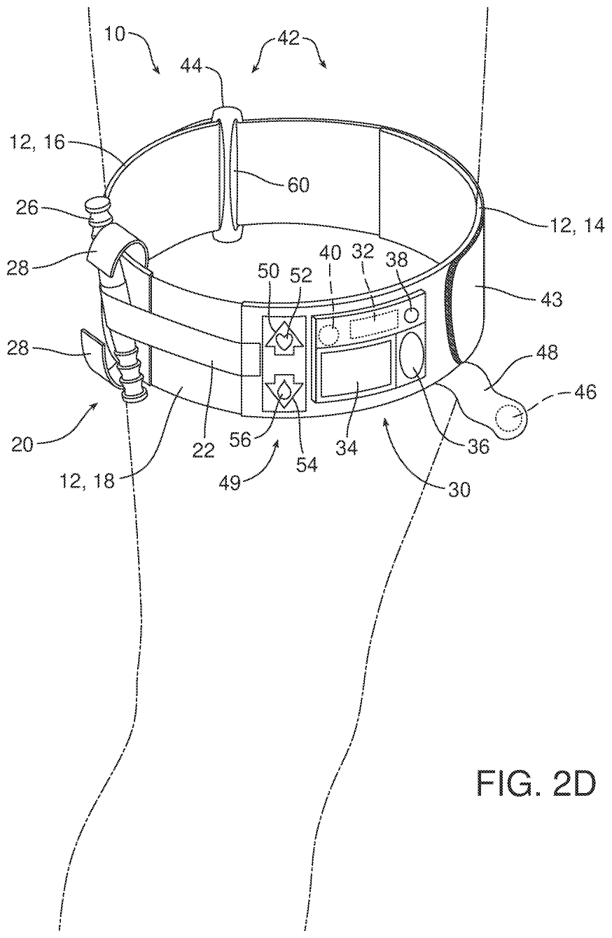

As illustrated in FIGS. 2A-2D, the tourniquet device 10 illustrated in FIG. 1 is useful in a method of occluding blood flow through a body part to which the tourniquet device is applied. Advantageously, the user is guided, by the controller 32, through each step of the tourniquet application process by audio and visual direction provided to the user through the information display 34 and the indicator 36. This ensures proper application of the tourniquet device 10 to the body part.

That method or process includes positioning the fastening strap 12 of the tourniquet device 10 around the body part and fastening the fastening strap in position. As shown in FIG. 2A, this is done by looping the fastening strap 12 around the body part, with reference to the indicia 49 which show which side of the fastening strap is oriented toward the heart and which side is oriented away from the heart. As shown in FIG. 2B, the free end of the first end section 14 of the fastening strap is then threaded or extended through the opening 60 in the fastening ring 44. The free end of the fastening strap 12 is then pulled until the fastening strap is snugged against the body part and then the cooperating hooks and loops of the conductive hook and loop fastener 43 are connected or fastened together.

This action closes an electrical circuit, formed by the conductive hook and loop fastener 43, that is monitored by the controller 32. Thus, the method includes detecting, by the controller 32, when the fastening strap 12 is properly fastened. The controller 32, in turn, causes the indicator 36 to provide an indication to the user that the fastening strap 12 has been properly positioned for use. This signaling may be an audio signal through a speaker, a visual signal by indicator lamp or through the information display or any combination of the above. This feedback confirmation gives the user confidence to proceed with the next step which is the tightening of the fastening strap 12 around the body part to occlude blood flow past the fastening strap.

In accordance with instructions provided by the controller 32 through the information display 34 and the indicator 36, the fastening strap 12 is then tightened by the user around the body part to occlude blood flow past the fastening strap. As shown in FIG. 2C, this is done by twisting and winding, by windlass/rod 26, the tightening strap. As a result, the two sections of the tightening strap 22, 24 are twisted together. This action serves to pull the two end sections 14,16 of the fastening strap 12 together, thereby squeezing the body part and gradually occluding the blood flow past the strap 12.

As the windlass/rod 26 is used to tighten the fastener strap 12 around the body part, the controller 32 monitors blood flow in the body part downstream from the fastener strap through operation and feedback received from the at least one sensor 46. Thus, the method includes detecting, by the at least one sensor 46, when blood flow is fully occluded. Once this occurs, the method includes alerting the user to quit tightening the fastening strap 12. This alert is provided by the controller 32 through operation of information display 34 and/or the indicator 36 to provide an audio and/or visual signal. This is followed by the controller 32 initiating a timer to monitor a duration of time since blood flow occlusion first occurred. That time duration may be displayed on the information display 34.

Once blood flow has been occluded, as confirmed by the controller 32 through the information display 34 and/or indicator 36, the method includes the step of engaging the windlass/rod 26 with the windlass retainer 28 (See FIG. 2D) to hold the windlass/rod in a wound condition and maintain the necessary pressure to continue the occlusion of blood flow past the tourniquet device 10. The controller 32 may provide a prompt to the user to complete this step through the information display 34 or a separate indicator 36.

In the event there is an interruption in blood flow occlusion as the result of some event, such as the moving of the individual to a different location, the controller 32 will provide: (a) a warning signal, in the form of an audio signal through a speaker, a visual signal by indicator lamp or through the information display 34 or any combination of the above, and (b) an indication of the time at which the blood flow occlusion was interrupted. The controller 32 will then advise the user, through the information display 34 and the indicator 36, how to restore blood flow occlusion by operation of the windlass/rod 26. Once blood flow occlusion is again restored by tightening the windlass/rod 26 and engaging the windlass with the retainer 28, the controller 32 will note the time of blood flow occlusion restoration and the duration of the same so that all relevant blood flow events are known to the subsequent care-giver.

In summary, the tourniquet device 10 and related method are characterized by numerous benefits and advantages including but not necessarily limited to:
a. a signal to the user when the fastening strap 12 has been properly positioned on the body part;
b. a signal directing the user to tighten the fastening strap;
c. a sensor 46 adapted to detect when arterial occlusion has occurred and blood flow past the fastening strap through the body part has ceased;
d. a signal to the user when to stop tightening the fastening strap;
e. a timer to monitor and indicate the duration of time blood flow has been occluded;
f. a signal to the user indicating any interruption in blood flow occlusion after blood flow occlusion has been established;
g. a timer to monitor and indicate the duration of any interruption to blood flow occlusion after blood flow occlusion has been established;
h. a signal to the user indicating when blood flow occlusion has been reestablished; and
i. a timer to monitor and indicate the duration of time blood flow occlusion has been reestablished.

In this way, it should be appreciated that the “smart” tourniquet device 10 provides real time assistance to the tourniquet user as the user provides medical aid to the injured individual in the field. As a result, the user is empowered to properly apply the tourniquet and occlude blood flow to the hemorrhage in the body part. Thus, the user can act confidently to save a life. In addition, the “smart” tourniquet device 10 provides critical blood flow information to the subsequent care-giver at a remote hospital or medical care facility. As a result, the care-giver has additional information upon which to treat the injured individual thereby providing the care-giver with the best opportunity to reduce trauma related morbidity and mortality.

This document may be said to relate to the following items.
1. A tourniquet device, comprising:
   a fastening strap;
   a tourniquet tightener spanning at least a portion of the fastening strap; and
   a control module carried on the fastening strap, the control module including a controller, an information display, and a power source.
2. The tourniquet device of item 1, wherein the tourniquet tightener includes a tightening strap connected across at least a portion of the fastening strap and a windlass adapted for winding the tightening strap to tighten the fastening strap around a body part and occlude blood flow through the body part of an individual.
3. The tourniquet device of item 1 or item 2, further including a fastener carried on the fastening strap, the fastener including a hook and loop fastener carried on a first end of the fastening strap and a fastening ring carried on a second end of the fastening strap.
4. The tourniquet device of item 3, wherein the hook and loop fastener is conductive.
5. The tourniquet device of item 4, further including at least one sensor adapted to detect occlusion of blood flow through the body part.
6. The tourniquet device of item 5, wherein the at least one sensor is a pulse sensor.
7. The tourniquet device of item 5, wherein the at least one sensor is an oxygen sensor.
8. The tourniquet device of item 5, wherein the controller is adapted to monitor a duration of time since blood flow occlusion and indicate the time duration on the information display.
9. The tourniquet device of item 1, further including an indicator wherein the indicator comprises (a) an audio speaker, (b) an indicator light or (c) an audio speaker and an indicator light that is adapted to be activated by the controller upon detection of a predetermined condition.
10. The tourniquet device of item 1, further including indicia carried on the fastening strap and adapted to indicate which side of the fastening strap is to be oriented toward a heart of the individual to which the tourniquet device is applied.
11. The tourniquet device of item 2, further including a windlass retainer carried on the fastening strap and adapted to hold the windlass in a wound condition.
12. A method of occluding blood flow through a body part using a tourniquet device, comprising:
   positioning a fastening strap of the tourniquet device around the body part and fastening the fastening strap in position;
   tightening the fastening strap around the body part to occlude blood flow past the fastening strap;
   detecting, by at least one sensor, when blood flow is occluded;
   alerting a user to quit tightening the fastening strap once blood flow is occluded; and
   initiating, by controller, a timer to monitor a duration of time since blood flow occlusion.
13. The method of item 12, further including detecting, by the controller, when the fastening strap is properly fastened.
14. The method of item 13, further including, signaling, by the controller, when the fastening strap is properly fastened.
15. The method of item 14, wherein the signaling when the fastening strap is properly fastened includes (a) providing an audio indication signal, (b) providing a visual indication signal or (c) providing both an audio indication signal and a visual indication signal.
16. The method of item 14, wherein the tightening of the fastening strap includes winding, by windlass, a tightening strap, carried on the fastening strap, to tighten the fastening strap around the body part.
17. The method of item 16, wherein the winding, by the windlass, includes turning a rod to twist and shorten the tightening strap.
18. The method of item 17, further including engaging the rod with a windlass retainer, carried on the tourniquet device, to hold the rod in a wound condition once the blood flow has been occluded.
19. The method of item 12, further including displaying the duration of time since blood flow occlusion on a display carried on the tourniquet device.
20. A tourniquet device, comprising:
   a fastening strap adapted for positioning around a body part;
   a tourniquet tightener carried on the fastening strap;
   a conductive fastener carried on the fastening strap; and
   a control module carried on the fastening strap, the control module including a controller adapted to monitor the conductive fastener and provide a signal when the conductive fastener has been properly fastened.
21. A method of occluding blood flow through a body part using a tourniquet device, comprising:
   monitoring and guiding, by a controller, proper application of the tourniquet device to the body part;
   detecting, by the controller, arterial occlusion in the body part; and
   alerting, by the controller, a user when the arterial occlusion has been achieved.
22. The method of item 21, further including initiating, by the controller, a timer to time a duration of the arterial occlusion.

Each of the following terms written in singular grammatical form: “a”, “an”, and “the”, as used herein, means “at least one”, or “one or more”. Use of the phrase “One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or, unless the context clearly dictates otherwise. For example, the phrase: “a tightening strap”, as used herein, may also refer to, and encompass, a plurality of tightening straps.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The phrase “consisting of”, as used herein, is closed-ended and excludes any element, step, or ingredient not specifically mentioned. The phrase “consisting essentially of”, as used herein, is a semi-closed term indicating that an item is limited to the components specified and those that do not materially affect the basic and novel characteristic(s) of what is specified. Terms of approximation, such as the terms about, substantially, approximately, etc., as used herein, refers to ±10% of the stated numerical value.

The foregoing has been presented for purposes of illustration and description. It is not intended to be exhaustive or to limit the embodiments to the precise form disclosed. Obvious modifications and variations are possible in light of the above teachings. For example, rather than including the indicia 49, the information display 34 could indicate which side of the fastening strap 12 should be oriented toward the heart and which side should be oriented away from the heart of the individual to which the tourniquet device 10 is being applied. As another example, the windlass retainer 28 may take substantially any form adapted to hold the windlass in place and prevent unwinding and release of pressure providing for the occlusion of blood flow. All such modifications and variations are within the scope of the appended claims when interpreted in accordance with the breadth to which they are fairly, legally and equitably entitled.

What is claimed:

1. A tourniquet device, comprising:
a fastening strap;
a tourniquet tightener spanning at least a portion of the fastening strap; and
a control module carried on the fastening strap, the control module including a controller, an information display, and a power source.

2. The tourniquet device of claim 1, wherein the tourniquet tightener includes a tightening strap connected across at least a portion of the fastening strap and a windlass adapted for winding the tightening strap to tighten the fastening strap around a body part and occlude blood flow through the body part of an individual.

3. The tourniquet device of claim 2, further including a fastener carried on the fastening strap, the fastener including a hook and loop fastener carried on a first end of the fastening strap and a fastening ring carried on a second end of the fastening strap.

4. The tourniquet device of claim 3, wherein the hook and loop fastener is conductive.

5. The tourniquet device of claim 4, further including at least one sensor adapted to detect occlusion of blood flow through the body part.

6. The tourniquet device of claim 5, wherein the at least one sensor is a pulse sensor.

7. The tourniquet device of claim 5, wherein the at least one sensor is an oxygen sensor.

8. The tourniquet device of claim 5, wherein the controller is adapted to monitor a duration of time since blood flow occlusion and indicate the time duration on the information display.

9. The tourniquet device of claim 1, further including an indicator wherein the indicator comprises (a) an audio speaker, (b) an indicator light or (c) an audio speaker and an indicator light that is adapted to be activated by the controller upon detection of a predetermined condition.

10. The tourniquet device of claim 1, further including indicia carried on the fastening strap and adapted to indicate which side of the fastening strap is to be oriented toward a heart of the individual to which the tourniquet device is applied.

11. The tourniquet device of claim 2, further including a windlass retainer carried on the fastening strap and adapted to hold the windlass in a wound condition.

12. A method of occluding blood flow through a body part using a tourniquet device, comprising:
positioning a fastening strap of the tourniquet device around the body part and fastening the fastening strap in position;
tightening the fastening strap around the body part to occlude blood flow past the fastening strap;
detecting, by at least one sensor, when blood flow is occluded;
alerting a user to quit tightening the fastening strap once blood flow is occluded; and
initiating, by controller, a timer to monitor a duration of time since blood flow occlusion.

13. The method of claim 12, further including detecting, by the controller, when the fastening strap is properly fastened.

14. The method of claim 13, further including, signaling, by the controller, when the fastening strap is properly fastened.

15. The method of claim 14, wherein the signaling when the fastening strap is properly fastened includes (a) providing an audio indication signal, (b) providing a visual indication signal or (c) providing both an audio indication signal and a visual indication signal.

16. The method of claim 14, wherein the tightening of the fastening strap includes winding, by windlass, a tightening strap, carried on the fastening strap, to tighten the fastening strap around the body part.

17. The method of claim 16, wherein the winding, by the windlass, includes turning a rod to twist and shorten the tightening strap.

18. The method of claim 17, further including engaging the rod with a windlass retainer, carried on the tourniquet device, to hold the rod in a wound condition once the blood flow has been occluded.

19. The method of claim 12, further including displaying the duration of time since blood flow occlusion on a display carried on the tourniquet device.

20. A tourniquet device, comprising:
a fastening strap adapted for positioning around a body part;
a tourniquet tightener carried on the fastening strap;
a conductive fastener carried on the fastening strap; and
a control module carried on the fastening strap, the control module including a controller adapted to monitor the conductive fastener and provide a signal when the conductive fastener has been properly fastened.
